(19) United States
(12) Patent Application Publication
(10) Pub. No.: US 2005/0070500 A1
(43) Pub. Date: Mar. 31, 2005

(54) Method of modulating release of saccharides and uses thereof

(76) Inventors: Isabelle Boucher l, Montreal Quebec (CA); Serge Brunet, Waterloo Quebec (CA)

Correspondence Address: OGILVY RENAULT, 1981 MCGILL COLLEGE AVENUE, SUITE 1600, MONTREAL, QC, H3A2Y3, CA

(21) Appl. No.: 10/498,002

(22) PCT Filed: Dec. 12, 2002
(86) PCT No.: PCT/CA02/01917

Related U.S. Application Data
(60) Provisional application No. 60/339,339, filed on Dec. 13, 2001.

(51) Int. Cl. A61K 31/737

(52) U.S. Cl. 514/54; 514/55

(57) ABSTRACT

The present invention relates to a method of controlled release of saccharides and oligosaccharides in human and animal. Polysaccharides are digested in a manner to provide oligomers having desired numbers of units of saccharides or monosaccharides, most particularly glucosamine and N-acetyl-glucosamine and derivatives thereof. The rate of release of monosaccharides is proportional to the length of the oligomers administered to an organism, and has targeted physiological effects depending on the length of the oligomers used.

### TECHNICAL FIELD

The invention relates to a method and composition for delayed delivery of chondroprotective, chondrosynthesis stimulating agents.

### BACKGROUND OF THE INVENTION

In recent years, many types of disorders/illnesses involving the destruction or decomposition of glycosaminoglycans (GAG) and proteoglycans (PG) appeared or became widespread. Most of these disorders stem from autoimmune responses in the body and provide symptoms related to inflammation and articular/protective mucous degradation. There is a growing body of evidence that suggests that supplementation with GAG constituents help reduce or eliminate the ailing symptoms.

In the past, it has been found that administering glucosamine (GS) or N-acetyl-D-glucosamine (NAG) to mammals reduced and rendered imperceptible the symptoms related to joint inflammation and articular degradation. Recently, glucosamine has been produced and marketed towards eliminating the same symptoms in humans. More specifically GS and in a lesser manner NAG have been found to offer substantial physical and emotional benefit against the ailments caused by the degradation of joint cartilage and mucosal tissues. It has been speculated that the onset of chondrodegenerative diseases may be caused by one or many factors involving environmental, nutritional, normal aging, genetic and stress factors.

More recently, other chondroprotective agents so as to supply the necessary building blocks for cartilage and mucosal membrane regeneration in an exogenous way or to viscosupplement the joints. Such agents are hyaluronic acid (HY), collagen (CO), chondroitin (CH), alone or in their salt forms.

Researches into the metabolism of glucosamine in dogs and humans have shown that gastrointestinal absorption is about 87% to 90% respectively. After absorption, glucosamine concentrates in the liver, where it is incorporated into plasma proteins, degraded into its smaller compounds or utilized for other biosynthetic processes (skin, connective tissue, and mucous membrane). After 144 hours, it is reported that, respectively in dogs and humans, about 10% and 11% of glucosamine is found in urine. Moreover, it was also noticed that a significant quantity of glucosamine was metabolized into CO₂ and water and expelled through the lungs. However, these studies did not include the precise measurement of this last parameter French Patent No. 2,473,887 discloses the use of biochemical precursors of glucosamineglycans for the treatment of vascular disorders of functional or organic origin in which there is insufficient blood flow to the limbs, for asphyxic hypoxydotic symptoms, and in cosmetology, for skin defects caused by insufficient circulation to the skin. The precursors, which include N-acetylglucosamine, increase the elasticity of perivascular tissue, resulting in an increase in arterio-capillary blood flow, without having a vasodilatating action.

U.S. Pat. No. 4,006,224 discloses the treatment of ulcerative colitis or regional enteritis in a mammal by administering monomers of D-glucosamine, or one of its salts. Equal or superior results to the conventional treatments of the two conditions are obtained. The dose is 20-300 mg/kg of D-glucosamine, HCl daily. In a clinical trial, a patient with Crohn's Disease that was not affected by ACTH or Prednisone™ was given D-glucosamine-HCl subcutaneously. The symptoms stopped after several weeks of treatment.

N-Acetylglucosamine and glucosamine units were considered for use in the prevention and treatment of degenerative joint diseases and cartilage loss, and were found to increase the glycosaminoglycans present in the cartilage to restore cartilage. Under monomeric form, glucosamine may also be considered in connection with arthritis and oral and injected glucosamine may be partially useful for arthrosic patients.

Among important potential sources of NAG and GS are chitin and chitosan. Chitin, a polysaccharide, is a biopolymer that occurs in nature in greater quantities than any other biopolymer except cellulose. Its most common occurrence is in the shells of crustaceans, e.g. crabs, shrimps and lobsters, where it occurs generally in admixture with mineral and proteinaceous material. Basically, chitin is a polymer of the N-acetyl glucosamine monomer unit, although some of the monomer units in its structure are devoid of the acetyl substituent and deacetylated chitin could more properly be said to be a copolymer of N-acetyl glucosamine and glucosamine monomers, with the latter constituting a variable proportion, generally from 1 to 20 percent of the monomer units, usually substantially 5 to 10 percent.

Chitosan is derived from chitin by removal of most of the acetyl substituents on the copolymer, usually by hydrolysis, to leave a copolymer having generally from 5 to 20 percent of N-acetyl glucosamine monomer units and correspondingly from 80 to 95 percent of glucosamine units in its structure.

Even if administration, mostly oral, of chitin, chitosan or monomers of their units, namely NAG and GS, has been performed, no application allowed yet an equilibrated exchange between the rate of the body to catalyze its reservoir of important biological molecules and the modulation of its capability to use it for different needs.

It would be highly desirable to be provided with a new mean of delayed or modulated releasing of saccharides in different metabolic disorders in animals.

### SUMMARY OF THE INVENTION

One object of the present invention is to provide a method of modulating release of monosaccharides in an animal which comprises the steps of:
treating a source of polysaccharides to produce oligomers of saccharides of desired length; and
administrating at least one of the oligomers as obtained from step a) or physiologically acceptable chemically, biochemically or biologically modified form of the oligomers to an animal in an amount sufficient to allow a modulated lasting release of the monosaccharides and obtain a physiological effect,
   wherein the lasting release lasts for a period of time proportional to the length of the oligomers, wherein more long is the oligomers, more long is the release of monosaccharides to be assimilated by the animal.

The polysaccharide source may be selected from the group of chitin, chitosan, hyaluronan, chondroitin, dermatan, keratan, and derivatives thereof.

The monosaccharide can be selected from the group consisting of a monomer of glucosamine, an N-acetylglucosamine (NAG), a galactosamine, an N-acetyl-galactosamine, a galactose, a glucuronate, and an iduronate, or salts derivative thereof.

Also, oligomers of saccharides, that can be qualified as being based prodrug, can be a tandem of at least one of glucosamine or N-acetylglucosamine.

The treatment for preparing the oligomers of saccharides is performed with at least one of enzymatic, biological, chemical, mechanical, or radioactive treatment.

The desired length of the oligomers of saccharides consists of between about 2 to 100 saccharides.

An oral administration, an intravenous administration, an intramuscular administration, an intraarticular administration, an intrasynovial administration, or a cutaneous application may perform the administrating of the oligomers of saccharides.

The physiological effect of glucosamine prodrugs is at least one of chondroregenerative or chondroprotective effect, prebiotic effect, probiotic effect, food additive effect, nutraceutical effect, wounding effect, immunomodulatory effect, systemic anti-inflammatory effect, bacteriostatic effect, anti-fungic effect, hypolipidemic effect, hypoglycemic effect, hypocholesterolemic effect, or anti-oxidant effect.

Another object of the present invention is to provide the use of an oligomer of saccharides in the manufacture of a medicament for the treatment of inflammation, or osteoarthritis.

In accordance with the present invention there is also provided a composition comprising an oligomer of saccharides having physiological effect selected from the group consisting of a chondroregenerative or a chondroprotective effect, a prebiotic effect, a probiotic effect, a food additive effect, a nutraceutical effect, a wounding effect, a immunomodulatory effect, an systemic anti-inflammatory effect, a bacteriostatic effect, an anti-fungic effect, a hypolipidemic effect, a hypoglycemic effect, a hypocholesterolemic effect, or an anti-oxidant effect, in association with a pharmaceutical or nutraceutical carrier.

The composition generally comprises a monosaccharide that may be selected from the group consisting of a monomer of a glucosamine, an N-acetylglucosamine (NAG), a galactosamine, an N-acetyl-galactosamine, a galactose, a glucuronate, and an iduronate, or salts derivative thereof.

Another object of the present invention is to provide a prodrug for modulating the in vivo release of a monosaccharide consisting of an oligomer of saccharides units consisting of between about 2 to 100 monosaccharides.

### BRIEF DESCRIPTION OF THE DRAWINGS

FIG. 1 illustrates mean (±standard deviation) serum glucosamine levels after oral capsule administration of 21.4 mg/kg nominal dose of glucosamine, Product X (composed of 2 up to 9 saccharides), and Product Y (composed of 2 up to 12 saccharides) in the acute post-dose period (0-2 hours);

FIG. 2 illustrates mean (±standard deviation) serum glucosamine levels after oral capsule administration of 214 mg/kg nominal dose of Product X, and Product Y in the acute post-dose period (0-2 hours);

FIG. 3 illustrates mean (±standard deviation) serum glucosamine levels after intravenous administration of 21.4 mg/kg nominal dose of Product X, and Product Y in the acute post-dose period (0-2 hours);

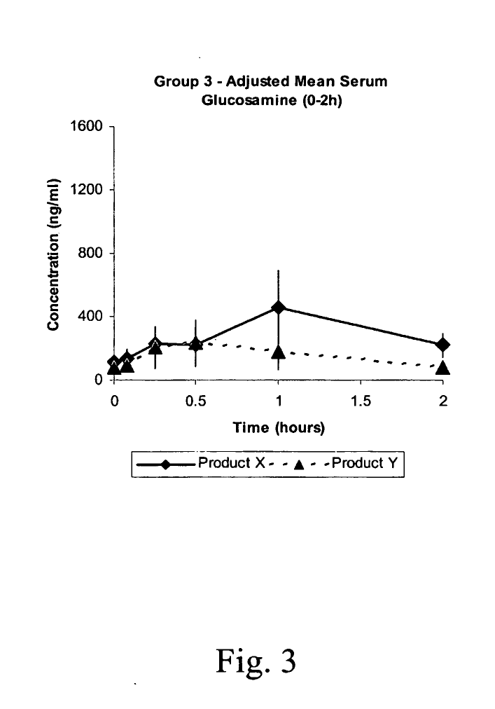

### DETAILED DESCRIPTION OF THE INVENTION

The present invention now will be described more fully hereinafter with reference to the accompanying drawings, in which preferred embodiments of the invention are shown. This invention, may, however, be embodied in many different forms and should not be construed as limited to the embodiments set forth herein; rather, these embodiments are provided so that this disclosure will be thorough and complete, and will fully convey the scope of the invention to those skilled in the art.

The following description is of a preferred embodiment by way of example only and without limitation to the combination of features necessary for carrying out the invention into effect. In accordance with the present invention, there is provided an oral administration of the GAG components, as for example, but not limited to, glucosamine, N-acetyl-glucosamine, galactosamine, N-acetyl-galactosamine, glucuronate, iduronate, galactose, alone or mixed, extracted from polysaccharides or biopolymers, such as chitin, chitosan, chondroitin, dermatan, keratan, hyaluronan, in their oligomer form, is advantageous for allowing a lasting release of monosaccharides and stimulating the growth and repairing connective tissue and articular cartilage. The oligosaccharide format of the said components is easily absorbed through the gastrointestinal tract and uptaken through the circulatory system.

The oligomers of oligosaccharide of the present invention provide an advantage as being the delivery vehicle for sustained or slow release of GAG components. Another advantage is that the oligomers of monosaccharides are metabolized more slowly compared to the monomers of monosaccharides. In this regard, the uptake of the raw components is reduced since the metabolic losses through urine, feces, breath and perspiration is less significant. There are multiple enzyme activities in the body fluids that can biodegrade the oligomers of monosaccharides and provide a sustained or slow release format for monomers to the different connective tissues and articular cartilage. The molecular ranges and sizes of the molecule uptake are important for the bioavailability and delivery kinetics.

In one embodiment of the present invention, there is provided a method of delivery into organism monomers of NAG and/or GS by the administration of short tandems containing between 2 to 100 units of NAG, monosaccharides, and/or GS in different proportions. It is intended with the invention to obtain targeted physiological effects by administering selected length of tandems of monosaccharides.

Preferably, the oligomers of the present invention comprise between about 2 to 25 saccharides.

According to different sources, other units of saccharides may consists of galactosamine, N-acetyl-galactosamine, galactose, glucuronate and iduronate, all products deriving from polysaccharides comprising among others, hyaluronan, chondroitin, dermatan and keratan.

One embodiment of the present invention is that administration or topical application of selected lengths of glucosamine or saccharide prodrugs may results in several targeted benefic health effects. Among these effects may be included a regenerative effect, most particularly for articular cartilage. Also, specific lengths of oligomers of monosaccharides may have a beneficial effect on mucosal membranes by inhibiting the inflammatory processes, as in the case of Crohn's disease, ulcerative colitis, or elsewhere by immunomodulating specific pathways of the immune system. Other length of oligomers of saccharides may have static or lethal effects on microorganisms, including bacteria and fungi.

Also, it is understood that administration or topical application of oligomers of saccharides of the present invention is intended to allow is modulated release of GS and/or NAG at a rate proportional to the length of the oligomers. By being naturally digested by endogenous enzymes, lysosyme, chitinase, N-acetyl-D-glucosaminidase of a treated organism, a human for example, the units of GS and/or NAG, or very small repetitions of GS and/or NAG comprising, for example but not limited to, 2 to 5 units, may be released as the oligosaccharides are digested.

Another embodiment of the invention is a mean by which GS (and in a lesser means NAG) supplementation helps to reduce and stop the degenerative aspects of the ailments. GS used for biosupplementation and chondrogenesis stimulation is normally supplied in the salt forms with the chloride or sulfate ions and administered orally by way of tablets, capsules or powders. In these salt forms, the glucosamine monomer active represents approximately 83% and 63% respectively of the total weight of the dried content. The usually recommended daily dosage ranges from 1.5 to 3.0 grams/day (3 to 6 capsules of 500 mg).

According to another embodiment of the invention, proposed use of NAG is as a source of amino sugar for the synthesis of molecules such as glycoproteins and glycosaminoglycans, which are rich in NAG and the synthesis of which is stimulated by NAG.

Another embodiment of the present invention is that an advantageous prolonged and sustained or slow release of monomers or short oligosaccharide chains can be achieved during in vivo or in situ digestion of oligomers of monosaccharides administered into or topically applied on a body.

Compositions containing the oligomers of the present invention may contain excipients adapted to all the forms used in creams or gels in pots or in tubes, milks, body emulsions, lotions in glass or plastic bottles and optionally in measuring bottles or also in jars, liquid soaps or bars.

Another object of the invention is to provide a dietary food supplement containing polysaccharides derivative oligosaccharides and an appetite modulating substance together with a nutriceutical to burn or better metabolize whatever fat the body does consume. The polysaccharide sources of the invention may be characterized as to the proportion of N-acetyl-D-glucosanine units and D-glucosamine units, and such is expressed as the degree of deacetylation of the fully acetylated precursor. The degree of deacetylation ranges preferably from 60% to 100%, more preferably from 90 to 100%, meaning that the proportion of N-acetyl-D-glucosamine units and D-glucosamine units in the oligomer is 0% to 40% of N-acetyl-D-glucosanine units and 60% to 100% of D-glucosamine units.

Although the preferred source of oligosaccharides is the deacetylated chitin and its derivative chitosan, other sources as hyaluronan, chondroitin, dermatan, keratan and derivatives thereof, may be considered in the present invention.

The physiologically functional derivatives also include prodrugs of the compounds according to the invention. Such prodrugs can be metabolized in vivo to give a compound according to the invention.

One embodiment of the present invention is to provide a method to release biologically active units of glucosamine including them as part of a soluble prodrug or oligomer of it. Oligomer prodrugs include chemical native or derivatives of a biologically active sub-unit, which, upon administration, eventually liberate the sub-unit in vivo. Oligomer prodrugs of the present invention allow modifying the onset and/or duration of action of a sub-unit in vivo and can modify the transportation, distribution or solubility of a drug, the sub-unit, in the body. Furthermore, oligomer prodrug formulations often reduce the toxicity and/or otherwise overcome difficulties encountered when administering pharmaceutical preparations. Oligomer prodrugs are often biologically inert or substantially inactive forms of the parent or active compound.

The present invention will be more readily understood by referring to the following examples that are given to illustrate the invention rather than to limit its scope.

### EXAMPLE I

### Pharmacokinetic Study

This study was undertaken to determine the safety of Products X and Y, and to determine the pharmacokinetic profiles of Products X, Y, and a commercially available oral glucosamine formulation (Glucosamine) following a single oral and intravenous administration to beagle dogs. The pharmacokinetic profiles were examined for indications of sustained or slow release of glucosamine by Products X and Y.

Materials and Methods

A total of nine (9) male Beagle dogs were used during this study. The animals were assigned to three treatment groups of 3 animals/group. Group 1 received low dose Products X, Y and Glucosamine by oral administration on Days 1, 8 and 15, respectively. Group 2 animals received high dose Products X and Y by oral administration on Days 1 and 8, respectively, and Group 3 animals received low dose Products X and Y by intravenous injection on Days 1 and 8, respectively. Low and high dose animals were administered nominal dose levels of 21.4 mg/kg and 214 mg/kg, respectively. These levels approximated 1× and 10× the standard daily oral glucosamine consumption (1500 mg) for an average human (70 kg standard body weight). The commercial glucosamine was assumed to contain 100% active ingredient.

The following parameters were recorded throughout the study: mortality, clinical signs, body weight and clinical pathology (hematology, coagulation and clinical chemistry). Serial blood samples were collected from all animals for pharmacokinetic profiling following each dosing.

Samples were collected via the jugular vein, at: 0 (pre-dose), 15, 30, 60, 120 minutes post-dose. A 5 minute post-dose sample was included for Group 3.

Safety Findings

No mortality was noted following the administration of the test articles.

No adverse clinical effects or effects on hematology, coagulation or clinical chemistry parameters were noted following oral capsule administration of Products X (21.4 and 214 mg/kg), Y (21.4 and 214 mg/kg), and commercially available Glucosamine (21.4 mg/kg).

Administration of both Products X and Y by intravenous injection resulted in an increase in ALP that is indicative of cartilage or bone formation. No adverse effects of intravenous administration were noted on coagulation parameters or other clinical hematology and chemistry parameters.

Results

Pre-Dose Glucosamine Levels

A serum sample was collected from all animals in all dose groups prior to dosing. The mean glucosamine level in the pre-dose samples was 113±62 ng/mL (n=9).

Oral Capsule Administration

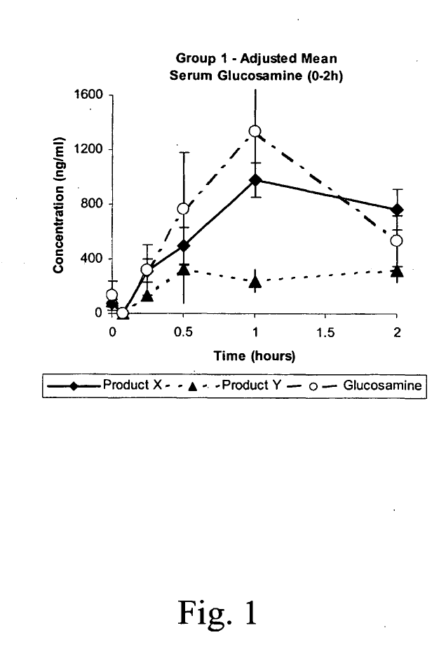

Following oral administration of commercial glucosamine (21.4 mg/kg), serum levels peaked at a group mean of 1337±359 ng/mL at 60 minutes post-dose, decreasing rapidly to 533±188 ng/mL by 120 minutes post-dose (FIG. 1).

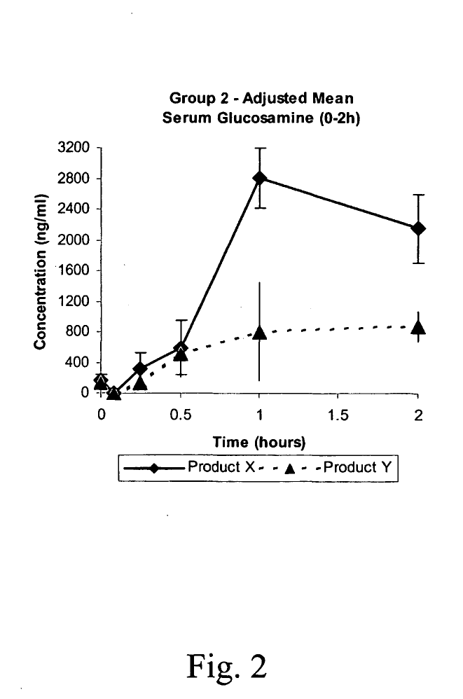

Following administration of Product X, at 60 minutes post-dose, glucosamine peaked at a group mean of 977±273 ng/mL and 2810±394 ng/mL in the 21.4 and 214 mg/kg dose groups, respectively, but had decreased somewhat to 761±315.5 ng/mL and 2153±452 ng/mL by 120 minutes post-dose (FIGS. 1 and 2). In contrast, as mentioned above, glucosamine administration of 21.4 mg/kg led to a peak mean serum glucosamine of 1337 ng/mL at 60 minutes and a rapid decline to 533 ng/mL by 120 minutes post-dose (FIG. 1).

Following administration of Product Y, glucosamine levels showed another pattern, with peak levels achieved anywhere from 30 to 120 minutes post-dose in the individual animals with mean levels of about 300 ng/mL and 800 mg/mL in the 21.4 and 214 mg/kg dose groups, respectively (FIGS. 1 and 2).

An area under the curve (AUC) analysis showed similar findings for the acute post-dose period (0-2 hours) with AUCs of 1397, 513, and 1651 ng/mL.hr for Product X, Y, and commercial glucosamine, respectively (Table 1). Thus, oral glucosamine, Product X, and Product Y deliver glucosamine into the serum in a ratio of 100:85:31 in the acute post-dose period, when compared on the basis of equivalent doses of 21.4 mg/kg glucosamine in each of the test articles. In addition, the AUC data show that Product Y delivers 37% of the glucosamine delivered by Product X in the first 120 minutes post-dose, in both the low and high dose groups (Table 1).

TABLE 1
Area Under the Curve Analysis for Products X, Y, and
Glucosamine From 0-2 Hours Post-Dosing

| | | Mean | Standard Deviation |
|---|---|---|---|
| Test Article | N | (mg/kg · h) | (mg/kg · h) |
| Group 1 | | | |
| Product X | 3 | 1397 | 413 |
| Product Y | 3 | 513 | 185 |
| Glucosamine | 3 | 1651 | 413 |
| Group 2 | | | |
| Product X | 3 | 3527 | 174 |
| Product Y | 3 | 1296 | 446 |
| Group 3 | | | |
| Product X | 3 | 626 | 281 |
| Product Y | 3 | 331 | 172 |

Taken together, the pattern of serum glucosamine levels indicate a smaller, but more sustained absorption of glucosamine into the blood in the 2 hours following administration of Product X and Y, when compared to the commercial preparation of glucosamine. The review of the raw peak and AUC data shows that glucosamine levels are lower following Products X and Y, indicating less bioavailability over the first 2 hours after dosing. In addition, glucosamine levels do not decrease as rapidly from peak levels following Products X and Y, indicating that higher serum levels are maintained for a longer period of time when compared to commercial glucosamine.

A comparison of the peak levels of serum glucosamine obtained for the low and high dose groups shows a 2.5-fold increase for both Product X and Y, despite a 10-fold increase in the dose administered. This non-linearity in the bioavailability of glucosamine from oral administration of Products X and Y may be due to saturation of absorption or catabolic pathways in the gut.

Intravenous Administration

Mean peak levels following intravenous administration were 458±495 for Product X and 205±132 for Product Y, much lower than those observed in Group 1, which received the same doses of Products X and Y by oral administration. The corresponding AUCs for the 0-2 hour period were also much lower in Group 3 (626 mg/kg.h for Product X; 331 mg/kg.h for Product Y). These data suggest that Products X and Y contain only a small amount of free glucosamine, and that additional factors such as catabolism of the test articles to glucosamine in the gut likely contribute to the larger glucosamine peaks observed following oral administration of Products X and Y.

The intravenous administration of Product Y produces a sustained increase in glucosamine that is consistent with a slow-release formulation.

### CONCLUSIONS

Administration of Product X, product Y, and a commercial source of Glucosamine (21.4 mg/kg) was well tolerated. No adverse clinical effects or laboratory values were noted.

Oral doses of Products X and Y up to 10× the recommended dose of glucosamine in humans were found to be well tolerated and appeared to be safe as used in this model.

Oral and intravenous administration of Product X and Y were found to increase serum glucosamine levels in the acute post-dose period (0-2 hours). Oral administration of Product X delivered about 3-fold more glucosamine than Product Y in the 2 hours immediately following dosing.

As expected, oral administration of commercial Glucosamine (21.4 mg/kg) also increased serum glucosamine in the acute post-dose period. However, the serum concentration of glucosamine after this treatment fell rapidly compared to effect measured after administration of Products X and Y.

A comparison of the glucosamine levels in the acute period for the low dose oral and intravenous routes of administration of both Product X and Product Y indicate that the oral route yields a higher acute serum glucosamine level, suggesting that metabolism by digestive processes or bacterial flora may enhance the bioavailability of the oral dose.

The data shows that oral and intravenous administration of both Products is associated with sustained elevations of serum glucosamine for up to 8-12 hours.

While the invention has been described in connection with specific embodiments thereof, it will be understood that it is capable of further modifications and this application is intended to cover any variations, uses, or adaptations of the invention following in general the principles of the invention and including such departures from the present disclosure as come within known or customary practice within the art to which the invention pertains and as may be applied to the essential features hereinbefore set forth, and as follows in the scope of the appended claims.

1. A method of modulating release of monosaccharides in a human or an animal comprising the steps of:
a) treating a source of polysaccharides to produce oligomers of saccharides of desired length; and
b) administrating at least one of said oligomers as obtained from step a) or physiologically acceptable chemically, biochemically or biologically modified form of said oligomers to a human or an animal in an amount sufficient to allow a lasting release of said monosaccharides and obtain a physiological effect,
wherein said lasting release lasts for a period of time proportional to the length of said oligomers.

2. The method according to claim 1, wherein said polysaccharide source is selected from the group of chitin, chitosan, hyaluronan, chondroitin, dermatan, keratan and derivatives thereof.

3. The method according to claim 1, wherein said polysaccharide source is chitosan.

4. The method according to claim 3, wherein said polysaccharide is deacetylated in proportion of between about 70 to 100%.

5. The method according to claim 3, wherein said polysaccharide is deacetylated in proportion of between about 80 to 100%.

6. The method according to claim 3, wherein said polysaccharide is deacetylated in proportion of between about 90 to 100%.

7. The method according to claim 1, wherein said monosaccharide is selected from the group consisting of a monomer of a glucosamine, a N-acetylglucosamine (NAG), a galactosamine, a N-acetyl-galactosamine, a galactose, a glucuronate, and an iduronate, or salts derivative thereof.

8. The method according to claim 1, wherein said oligomer of saccharides is a tandem of at least one of glucosamine or N-acetylglucosamine.

9. The method according to claim 1, wherein said treatment of step a), is at least one of enzymatic, biological, chemical, mechanical, or radioactive treatment.

10. The method according to claim 1, wherein said desired length of said oligomers of saccharides is between 2 to 100 monosaccharides.

11. The method according to claim 1, wherein said desired length of said oligomers of saccharides is between 2 to 25 monosaccharides.

12. The method according to claim 1, wherein said administrating of step b) is an oral administration, an intravenous administration, an intramuscular administration, an intraarticular administration, an intrasynovial administration, or a cutaneous application.

13. The method according to claim 1, wherein said physiological effect of step b) is at least one of chondroregenerative or chondroprotective effect, prebiotic effect, probiotic effect, food additive effect, nutraceutical effect, wounding effect, immunomodulatory effect, systemic anti-inflammatory effect, bacteriostatic effect, anti-fungic effect or anti-oxidant effect.

14. Use of an oligomer of saccharides in the manufacture of a medicament capable of modulated lasting release of a monosaccharide for the treatment of systemic inflammation.

15. The use according to claim 14, wherein said monosaccharide is selected from the group consisting of a monomer of a glucosamine, a N-acetylglucosamine (NAG), a galactosamine, a N-acetyl-galactosamine, a galactose, a glucuronate, an iduronate, and salts derivative thereof.

16. The use according to claim 14, wherein said oligomer of saccharides is a tandem of at least one of glucosamine or N-acetylglucosamine.

17. The use according to claim 14, wherein said desired length of said oligomer of saccharides is between 2 to 100 oligosaccharides.

18. The use according to claim 14, wherein said systemic inflammation is osteoarthritis.

19. A composition comprising an oligomer of saccharides modulating the release of monosaccharides having physiological effect selected from the group consisting of a chondroregenerative or a chondroprotective effect, a prebiotic effect, a probiotic effect, a food additive effect, a nutraceutical effect, a wounding effect, a immunomodulatory effect, an systemic anti-inflammatory effect, a bacteriostatic effect, an anti-fungic effect or an anti-oxidant effect, in association with a pharmaceutical or nutraceutical carrier.

20. The composition according to claim 19, wherein said monosaccharide is selected from the group consisting of a monomer of a glucosamine, a N-acetylglucosamine (NAG), a galactosamine, a N-acetyl-galactosamine, a galactose, a glucuronate, and an iduronate, or salts derivative thereof.

21. The composition according to claim 19, wherein said oligomer of monosaccharides is a tandem of at least two of glucosamine or N-acetylglucosamine.

22. The composition according to claim 19, wherein said desired length of said oligomer of saccharides is between 2 to 100 oligosaccharides.

23. The method according to claim 19, wherein said desired length of said oligomers of saccharides is between 2 to 25 monosaccharides.

24. The composition according to claim 19, wherein said inflammation is osteoarthritis.

25. A prodrug for modulating the in vivo release of a monosaccharide consisting of an oligomer of saccharides units consisting of between about 2 to 100 monosaccharides.

26. A prodrug for modulating the in vivo release of a monosaccharide consisting of an oligomer of saccharides units consisting of between about 2 to 25 monosaccharides.
